(19) United States
(12) Patent Application Publication
(10) Pub. No.: US 2015/0305750 A1
(43) Pub. Date: Oct. 29, 2015

(54) FOAM OCCLUSION DEVICE

(71) Applicant: Cook Medical Technologies LLC, Bloomington, IN (US)

(72) Inventors: Rick Hadley, Otterbein, IN (US); Andrew P. Isch, West Lafayette, IN (US); Rita Hadley, Otterbein, IN (US)

(21) Appl. No.: 14/698,189

(22) Filed: Apr. 28, 2015

Related U.S. Application Data
(60) Provisional application No. 61/985,178, filed on Apr. 28, 2014.

(51) Int. Cl. A61B 17/12

(52) U.S. Cl. CPC A61B 17/12159; A61B 17/12109; A61B 17/12168; A61B 17/1215; A61B 2017/12095

(57) ABSTRACT

An occlusion device comprising a plurality of expandable hydrophilic foam elements is described. When the device is deployed to a body vessel or a body cavity, it expands radially due to uptake of liquid from the body fluids with which it is in contact. The device may optionally have a surrounding meshwork, an inner spring, and a screw-type deployment mechanism. A method of deploying the device is also provided.

### BACKGROUND OF THE INVENTION

1. Field of Invention

The present invention relates to medical devices. More particularly, the invention relates to an occlusion device for occluding a lumen of a body vessel.

2. Background

Vascular occlusion devices are surgical implants that are placed within the vascular system of a patient. There are a number of reasons why it may be desirable to occlude a vessel. For example, the site of a stroke or other vascular accident can be treated by placing an occlusion device proximal of the site to block the flow of blood to the site, thereby alleviating leakage at the site. An aneurysm can be treated by the introduction of an occlusion device through the neck of the aneurysm. Tumours can be treated by occluding the flow of blood to a targeted site of interest.

Several known occlusion devices include metal coils which are capable of being deployed into a body vessel or body cavity. In some cases, occlusion can be achieved with the deposition of a single coil, but in other cases, multiple coils must be deployed to the occlusion site, prolonging the procedure. Furthermore, it cannot always be predicted how many coils may need to be introduced to a particular site. In some cases, the coils are made of expensive materials, such as platinum, thereby increasing both the cost and complexity of such procedures.

Contrarily, a device made of a self-expanding and space-filling material, particularly a hydrophilic material which takes on water from bodily fluid and swells to fill a vessel, would have the capacity to expand and occlude quickly and without the drawbacks of metal devices.

There is a need for a single device that can be deployed to the body cavity or body vessel to be occluded to affect occlusion in a single step and to do so with a space-filling, pliable material.

### BRIEF SUMMARY OF THE INVENTION

One embodiment of an occlusion device generally comprises a coil spring having a proximal end and a distal end, defining a longitudinal axis; a proximal collet disposed to the proximal end and a distal collet to the distal end; at least one expandable element disposed radially about a portion of the coil spring; and a covering disposed on the proximal collet about the proximal end, the covering extending from the proximal collet about the coil spring and the at least one expandable element, the covering being disposed on the distal collet about the distal end.

In another embodiment, an assembly for occlusion of a body vessel is provided, the assembly including an outer sheath having a tubular body comprising an open end, the tubular body including a lumen formed through the open end. The assembly also includes an inner member disposed within the sheath lumen and movable rotationally and longitudinally relative to the outer sheath; and an occlusion device comprising a coil spring having a proximal end and a distal end, defining a longitudinal axis. The occlusion device also includes a proximal collet disposed to the proximal end and a distal collet to the distal end. The occlusion device includes at least one expandable element disposed radially about a portion of the coil spring; and a covering disposed on the proximal collet about the proximal end, the covering extending from the proximal collet about the coil spring and the at least one expandable element, the covering being disposed on the distal collet about the distal end. The occlusion device is disposed within the lumen and removably coupled to the distal end of the inner member and deployable through the open end of the outer sheath.

In another embodiment, a method method of occluding a body vessel is provided. The method includes a first step of wetting an occlusion device, the occlusion device comprising a coil spring having a proximal end and a distal end, defining a longitudinal axis; a proximal collet disposed to the proximal end and a distal collet to the distal end; at least one expandable element disposed radially about a portion of the coil spring; and a covering disposed on the proximal collet about the proximal end, the covering extending from the proximal collet about the coil spring and the at least one expandable element, the covering being disposed on the distal collet about the distal end. In a second step, the method includes crimping the expandable element of the occlusion device to dispose the occlusion device within a lumen. In a third step, the method includes loading the occlusion device into the lumen. In a fourth step, the method includes introducing the delivery assembly percutaneously into the body vessel. In a fifth step, the method includes delivering the occlusion device to the body vessel.

Further objects, features, and advantages of the present invention will become apparent from consideration of the following description and the appended claims when taken in connection with the accompanying drawings.

### BRIEF DESCRIPTION OF THE DRAWINGS

FIG. 1 is side view of an occlusion device in accordance with the teachings of the present invention;

FIG. 2 is a perspective view of an expandable element for an occlusion device in accordance with the teachings of the present invention;

FIG. 3A is a perspective view of an occlusion device in accordance with another embodiment of the present invention;

FIG. 3B is a view of a screw and spring assembly in accordance with an embodiment of the present invention;

FIG. 4 is a perspective view of an occlusion device in accordance with another embodiment of the present invention;

FIG. 5 is a side view of an occlusion device in accordance with another embodiment of the present invention;

FIG. 6A-B are exploded views of a delivery assembly to be used in conjunction with an occlusion device; and

FIG. 7A-F are illustrations of the steps of preparation of the device for loading and delivery to a body vessel of a patient in accordance with various embodiments of the invention.

### DETAILED DESCRIPTION OF THE INVENTION

The description that follows is not intended to limit the scope of the invention in any manner, but rather serves to enable those skilled in the art to make and use the invention.

It is to be understood that the figures are schematic and do not show the various components to their actual scale. In many instances, the figures show scaled up components to assist the reader. In other cases, the figures may display components of devices with additional space therebetween so as to illustrate all portions of each component.

In this description, when referring to a device, an introducer, or deployment assembly, the term distal is used to refer to an end of a component which in use is furthest from the physician during the medical procedure, including within a patient. The term proximal is used to refer to an end of a component closest to the physician and in practice in or adjacent an external manipulation part of the deployment or treatment apparatus. Similarly, when referring to an implant such as an occlusion device the term distal is used to refer to an end of the device which in use is furthest from the physician during the medical procedure and the term proximal is used to refer to an end of the device which is closest to the physician during the medical procedure.

The terms “substantially” or “about” used herein with reference to a quantity includes variations in the recited quantity that are equivalent to the quantity recited, such as an amount that is equivalent to the quantity recited for an intended purpose or function.

As used herein, the term “biocompatible” refers to a material that is substantially non-toxic in the in vivo environment of its intended use, and that is not substantially rejected by the patient's physiological system or is non-antigenic. This can be gauged by the ability of a material to pass the biocompatibility tests set forth in International Standards Organization (ISO) Standard No. 10993; the U.S. Pharmacopeia (USP) 23; or the U.S. Food and Drug Administration (FDA) blue book memorandum No. G95-1, entitled “Use of International Standard ISO-10993, Biological Evaluation of Medical Devices Part-1: Evaluation and Testing.” Typically, these tests measure a material's toxicity, infectivity, pyrogenicity, irritation potential, reactivity, hemolytic activity, carcinogenicity, immunogenicity, and combinations thereof. A biocompatible structure or material, when introduced into a majority of patients, will not cause a significantly adverse, long-lived or escalating biological reaction or response, and is distinguished from a mild, transient inflammation which typically accompanies surgery or implantation of foreign objects into a living organism.

The present disclosure generally provides an occlusion device which can be used with any suitable occlusion device delivery system by a physician to deliver an occlusion device into a body vessel or a cavity of a patient, such as a renal artery.

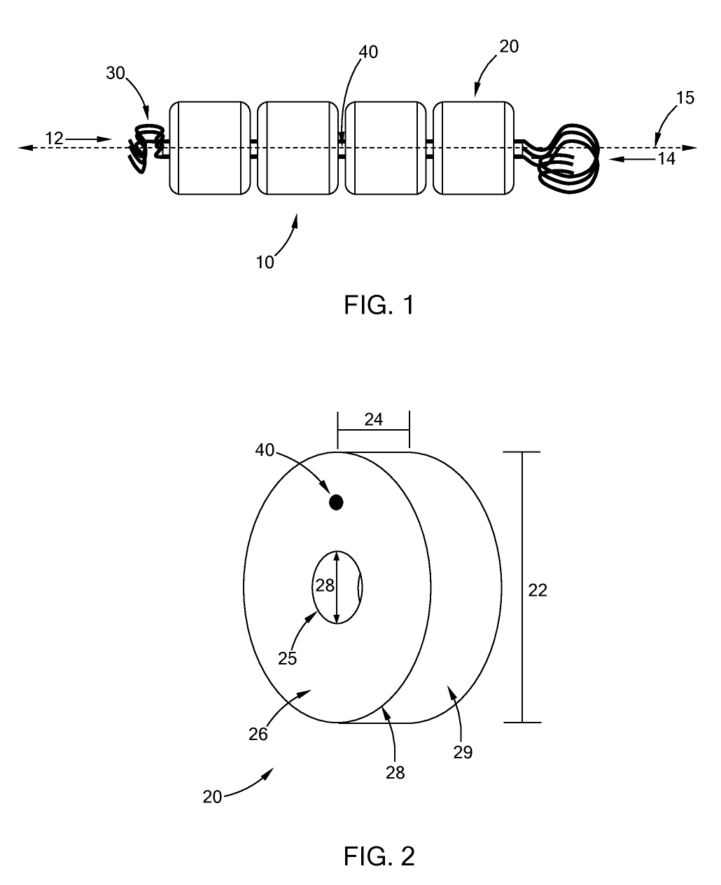

Referring to FIG. 1, one embodiment of an occlusion device 10 is depicted. The occlusion device 10 has a proximal end 12 and a distal end 14, and generally comprises a plurality of expandable elements 20. In one embodiment, the expandable elements 20 may comprise hydrophilic foam material. In another embodiment, an expandable element may be a sponge or made of a sponge material. The hydrophilic expandable elements 20 are connecting by a connecting member, in this case a plurality of sutures 30. Radiopaque members 40 are also included in the device. As depicted in FIG. 1, the radiopaque members are positioned in gaps that occur between hydrophilic expandable elements 20, but other locations are suitable, including along the lengths of the sutures 30 or embedded within the hydrophilic expandable elements 20 themselves. Longitudinal axis 15 runs through the body of the device.

The device 10 could be used to provide temporary occlusion or permanent occlusion. It can also optionally include protruding elements at its exterior to irritate a vessel wall and cause restenosis, which will allow for a means of closure of the vessel.

The hydrophilic expandable elements 20 are capable being wetted by an aqueous fluid, such as water or a body fluid. When this wetting occurs, the hydrophilic foam elements 20 expand. Conversely, when the liquid is made to exit the foam elements, they dry and contract in size. The foam elements 20 are compressible. Prior to the embolization procedure, the compressible hydrophilic foam elements are placed inside of a delivery assembly in this dried, compressed state. After ejection from the delivery assembly, the foam elements 20 expand to provide for mechanical fixation of the device within the target body vessel, thereby providing occlusion of the vessel.

The hydrophilic foam elements may be made of expandable polyvinyl alcohol (PVA) foam material. They may have a compressible, porous structure. In one aspect, the expandable material is formalin crosslinked PVA foam. Alternatively, the particle 10 can be made of polyurethane.

In another aspect, the expandable element 20 can have any shape with a circular profile configured to provide a sealed occlusion with respect to the body vessel or cavity in which it is to be positioned. For example, the foam elements 20 according to one embodiment can be cylindrical. The initial configurations of these expandable elements are in the form of a scaled-down physical model of the site to which they are to be delivered. The elements are compressible into a compressed configuration that fits within a delivery catheter. To treat an aneurysm, the device 10 would not be placed mainly or exclusively within the aneurysm, leaving the vessel off of which the aneurysm originated open for blood flow. Instead, the size of the neck of the aneurysm would be estimated by an interventionalist and a device having a length greater than the neck of the aneurysm would be employed for insertion into the vasculature of the patient. One way of achieving this objective would be to select a device constructed of a plurality of expandable elements 20 such that there would be a middle portion equal in length to the length of the aneurysm neck, plus at least one additional expandable element 20 proximal to this middle portion and at least one additional expandable element 20 distal to the middle portion. This would allow for at least one expandable element 20 to be in a position to expand to the normal-sized diameter of the vessel on either side of the aneurysm, affecting substantially complete occlusion of the vessel proximal and distal to the neck of the aneurysm. After blood flow is stopped by the device 10, filling the space created by the aneurysm would no longer be a concern as the pressure from the expandable elements 20 on either side of the aneurysm would prevent further influx of fluid through the occluded vessel. The expandable elements 20 of the middle portion of the device would likely maintain contact with the healthy, unexpanded portions of the vessel wall opposite the aneurysm neck, and would likely expand to fill a part of the aneurysm itself, but would not expand to fill the entire aneurysm.

The hydrophilic expandable elements 20 may also be made of a hydrophilic, macroporous, polymeric, hydrogel foam material. In one example, this can be a swellable foam matrix formed as a macroporous solid comprising a foam stabilizing agent and a polymer or copolymer of a free radical polymerizable hydrophilic olefin or alkene monomer cross-linked with up to about 10% by weight of a multiolefin-functional cross-linking agent. Such polymeric hydrogels can have a high capacity to absorb and retain water, while the cross-link network prevents dissolution of the individual chains. The high water content, rubbery consistency, low toxicity and low interfacial tension make hydrogels resemble, to some degree, natural tissues.

Hydrogels from PVA units can provide mechanical strength without the need for a cross-linking agent, which may have an adverse effect when implanted. The integrity of the hydrogel material can be primarily derived from hydrogen bonding and the large number of small crystallites. Because of the high tensile strength of the PVA hydrogels, they may be manufactured into thin but strong devices.

Continuing with the description of FIG. 1, sutures 30 for linking the hydrophilic expandable elements 20 of the occlusion device 10 to one another may be made from a variety of suture types, including braided or monofilament sutures. Sutures 30 may be made from polyester, polypropylene, polyglycolic acid, polytetrafluoroethylene (PTFE), small intestinal submucosa, nylon, silk or any of a variety of absorbable or nonabsorbable suture materials known in the art.

The sutures 30 may be treated or coated with radiopaque materials to facilitate visualization of the device by radiography or fluoroscopy. The sutures 30 may also be coated with antibiotics or other antimicrobial agents, or may also be coated with other molecules to be delivered, such as for example clotting factors.

The device of FIG. 1 also comprises radiopaque members 40 for visualizing the device by fluoroscopy or radiography during implantation. Radiopaque material may be used in device 10 as discrete particles (as illustrated in FIG. 1) or, for example, as a coating over some or all of the device.

In the embodiment illustrated in FIG. 1, the radiopaque members 40 are each individually placed between the foam elements 20 and are held in place by any known means, including connecting to the sutures 30. The radiopaque members 40 are not interconnected with one another in any fashion; the embodiment of FIG. 1 does not, for instance, represent a single radiopaque core element running through a series of foam discs, but instead illustrates four foam elements 20 interposed between five radiopaque members 40.

Radiopaque materials to be used are non-toxic materials capable of being monitored or detected during injection into a mammalian subject by, for example, radiography or fluoroscopy. The radiopaque material may be either water soluble or water insoluble. Examples of water soluble radiopaque materials include metrizamide, iopamidol, iothalamate sodium, iodomide sodium, and meglumine. Examples of water insoluble radiopaque materials include tantalum, tantalum oxide, and barium sulfate, which are commercially available in the proper form for in vivo use. The radiopaque material may be a polymer, ceramic, or a noble metal. Examples of noble metals include gold, platinum, iridium, palladium, or rhodium, or a mixture thereof. The radiopaque material allows for better positioning and tracking of the device by fluoroscopy during delivery. In addition, the radiopaque materials can be incorporated in the closure device or assembly components by a variety of common methods, such as adhesive bonding, lamination between two material layers, and vapor deposition.

FIG. 2 is a close-up view of a hydrophilic foam element 20 according to one aspect of the invention. In this case, the element 20 has a circular disc shape. The element has a height or diameter 22 and a thickness or width 24. The disc has a center and at the center in this embodiment is hole 25 having hole diameter 28. The element 20 has a first face 26 and on the opposite side of the device is second face 27. The disc has a side portion 29 which has for its magnitude the value of width 24.

The hydrophilic foam elements may also be of other polygonal shapes and still affect occlusion of the vessel to be treated. Exemplary polygonal shapes include, but are not limited to triangle, quadrilateral, square, pentagon, hexagon, octagon, and the like. Circular shapes include circle, oval, ellipse, and the like.

As mentioned previously, the hydrophilic foam elements 20 expand upon contact with an aqueous fluid. Thus, when water or a body fluid comes in contact with a disc-shaped element 20 as show in FIG. 2, the disc will undergo an expansion in all dimensions. Of note is the expansion in the radial direction. The expansion of a disc element 20 results in a greater value for height or diameter 22 and thickness 24. Such radial expansion assists in occlusion of the body vessel and retention of the device by allowing it to become in contact with and patent with the vessel wall.

The expansion of such hydrophilic foam elements is gradual but fast. In cases where full occlusion is desired, this combination of features is advantageous, as fast occlusion permits the deployment procedure to proceed quickly, but gradual expansion allows for repositioning of the device if necessary; that is, the device expands quickly, but not so quickly that it is irreversibly seated in the lumen of the vessel it is to occlude. Quicker expansion minimizes risk of migration of the device through the vasculature, while gradual expansions minimizes acute forces experienced by the vessel walls, minimizing trauma thereto as a result of forceful expansion.

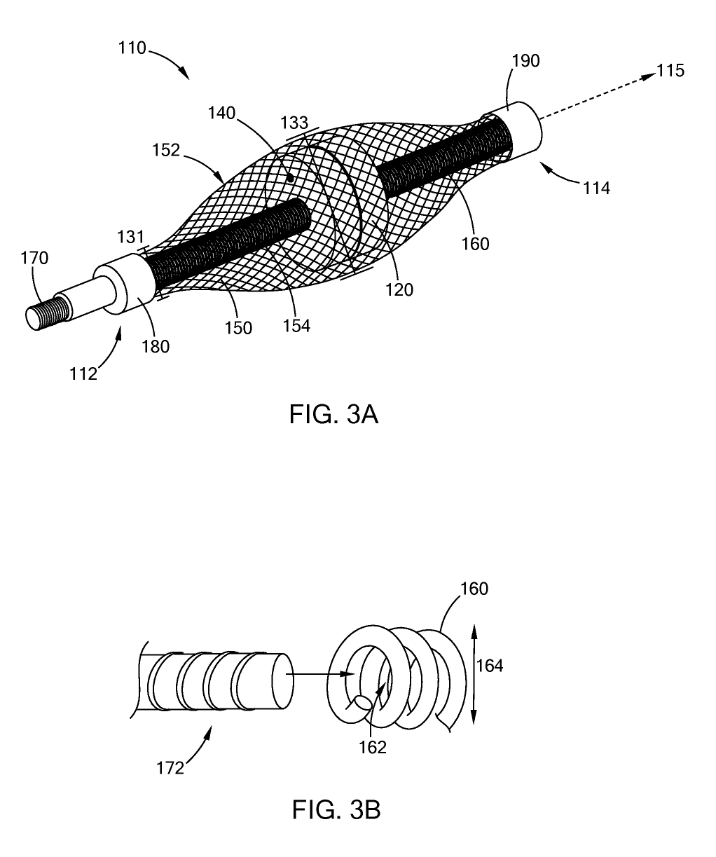

FIG. 3A illustrates another embodiment in accordance with the principles of the present invention. FIG. 3A shows the occlusion device 110 with expandable foam elements 120 contained inside of covering 150. The covering has an outer surface 152 and a covering lumen 152, where the foam elements 120 are contained.

The covering 150 may comprise a number of different constructions. For instance, the covering 150 may be a mesh comprising a metal. It may also be made of a polymer or of a textile material. The covering may be a sleeve with an open first end and an open second end, each of which are housed within a respective collet. The covering may be disposed radially around the coil spring and within the collet.

In one embodiment, the covering 150 may comprise a shape memory metal. In another embodiment, the covering 150 may comprise a polymer such as a polyethylene. In still another embodiment, the covering may comprise strands of polyethylene as well as strands of metal. In some embodiments, the metal may be a shape memory metal.

The covering 150 may can have virtually any textile construction, including weaves, knits, braids, filament windings and the like. A variety of textile constructions may be employed. With respect to weaves, any known weave pattern in the art, including simple weaves, basket weaves, twill weaves, velour weaves and the like may be used.

If a metal is employed in the covering 150, suitable materials include, for example, a superelastic material, a nickel-based superalloy, stainless steel wire, cobalt-chromium-nickel-molybdenum-iron alloy, cobalt chrome-alloy, stress relieved metal (e.g., platinum), or nickel-based superalloys, such as Inconel. The covering 150 may be formed of any appropriate material that will result in a self-expanding device capable of being percutaneously inserted and deployed within a body cavity, such as shape memory material.

Shape memory materials or alloys have the desirable property of becoming rigid, i.e., returning to a remembered state, when heated above a transition temperature. A shape memory alloy suitable for the present invention is nickel-titanium (NiTi) available under the more commonly known name Nitinol. When this material is heated above the transition temperature, the material undergoes a phase transformation from martensite to austenite, such that the material returns to its remembered state. The transition temperature is dependent on the relative proportions of the alloying elements Ni and Ti and the optional inclusion of alloying additives. The Nitinol could be of various types, such as linear elastic Nitinol or radiopaque Nitinol.

The shape memory metal could be configured in one embodiment to be pliable when loading into the delivery apparatus but spring to a straightened or expanded state when deployed in a body cavity. In one embodiment, the shape memory metal can have a transition temperature that is slightly below normal body temperature of humans, which is about 98.6° F. Thus, when the device 110 is deployed in a body vessel and exposed to normal body temperature, the alloy of the covering 150 will transform to austenite, that is, the remembered state, which for one embodiment of the present invention is the expanded state when deployed in the body vessel.

In contrast, to load the device 110 into the delivery apparatus, the device is cooled actively or passively to below human body temperature which will transform the material to martensite, which is more ductile than austenite, making the covering more malleable. As such, the device 110 can be more easily collapsed and pulled into a delivery apparatus.

The covering 150 may in some embodiments consist solely of or partly of a polymer. One type of polymer is a polyester, such as polyethylene terephthalate, known by its trade name of DACRON. DACRON is a colorless, semi-crystalline resin which can be semi-rigid to rigid. It is lightweight with high structural integrity. It is well-tolerated by the body when used in implantable devices. Significantly, DACRON is known as a thrombogenic material, which speeds occlusion by causing thrombus formation at the side of deployment of the occlusion device and impeding flow of fluids such as blood through the vessel. Other polymers with similar characteristics are well-suited for inclusion in the covering 150.

The covering layer may optionally contain thrombogenic fibers in order to speed occlusion and encourage thrombus formation. Thrombogenic fibrous materials include synthetic or natural fibrous material having thrombogenic properties. Exemplary thrombogenic fibrous materials include, but are not limited to, DACRON, cotton, silk, wool, polyester thread and the like.

The device of FIG. 3A has a proximal end 112 and a device body extending to a distal end 114. Longitudinal axis 115 runs through device 110. The device 110 has an overall elliptical shape due to its relatively narrow proximal end 112 and distal end 114 and expansion toward the center of the device as measured along its length. The covering 150 surrounds and encompasses the hydrophilic foam elements 120 and defines a first device portion height 131 toward proximal end 112 where it meets proximal collet 180. The proximal collet 180 and the distal collet are each open, hollow caps which have an opening which can accommodate the coil spring and the covering, or mesh, when disposed about the coil spring. The device 110 has its maximum height toward the center of the device as determined along the longitudinal axis 115 at second device portion height 133. In the embodiment illustrated, the radiopaque members 140 are embedded within the foam elements 120, but these radiopaque members could also be interspersed between the foam elements 120 or along the covering 150 among other locations.

As illustrated in FIG. 3A, the device 110 has two disc-shaped hydrophilic foam elements 120. However, the device could also be constructed with two, three, four, five, six, seven, eight, or more hydrophilic foam elements 120. It is also possible to construct the device with a single foam element 120.

Inner coil spring 160 passes through the holes of the disc-shaped expandable hydrophilic foam elements 120 in the device 110 of FIG. 3A. This spring is configured to contract in the absence of an outside pressure or stretching force. It extends through covering lumen 154 from proximal collet 180 to distal collet 190. The distal end of the inner coil spring 160 may be fixed to the distal collet 190 by any known method, or the proximal end of the inner coil spring 160 may be fixed to the proximal collet 180 by any known means, or both ends may be fixed to both collets in order to facilitate an operator's ability to increase or decrease tension on the inner coil spring 160.

The device of FIG. 3A is expanded by a screw mechanism. Threaded capture 170 on the proximal end of the device 110 can be attached to the delivery mechanism and allows the practitioner to control expansion of the device by rotating a member of the delivery system such as a pusher or an outer catheter. This threaded capture 170 also functions to assist in release of the device from the delivery assembly. The threaded capture 170 may extend proximally from the proximal end of the proximal collet 180 of a device according to the principles of the present invention. Rotation of the screw mechanism, such as for example a clockwise rotation, increases the tension on the inner coil spring 160, shortening the overall length of the device, and rotation in the opposite direction, in this instance a counterclockwise rotation, would reduce strain on the spring, expanding the overall length of the device. Shortening the device length increases the diameter of the outer covering 150 and expands the profile of the device in the radial dimension. Releasing spring tension and increasing the length of the device in the axial dimension decreases the profile of the device in the radial dimension and thus facilitates recapture and resheathing of a deployed or partially-deployed device should a practitioner wish to reposition the device.

FIG. 3B shows a screw mechanism in accordance with one embodiment of the present invention. The inner coil spring 160 is divided into two halves, one of which terminates in a threaded end 172. The other spring half has an inner diameter 164 of inner lumen 162 which is defined by the interior of the coils of the spring 160. By rotating the delivery apparatus component or components as described above, the practitioner deploying the device 110 can either bring the two device halves together or drive them apart depending on which direction the rotation is made. The threaded end 172 fits within the lumen 162 of the opposite spring half and becomes threadedly attached to the opposite spring half.

Alternately, both spring halves can simply comprise the spring coils themselves. In a device constructed in this way, the screw-type mechanism would work as the spirals of the coils would intermesh with one another, bringing the halves together without the use of any sort of extra threaded portion. The distal end of the proximal-most spring half in such an embodiment would be interposed within a space between the coils of the distal-most spring. Rotation of the proximal coil spring by an interventionalist would cause the proximal spring to move through the windings of the distal spring, further engaging the two spring halves.

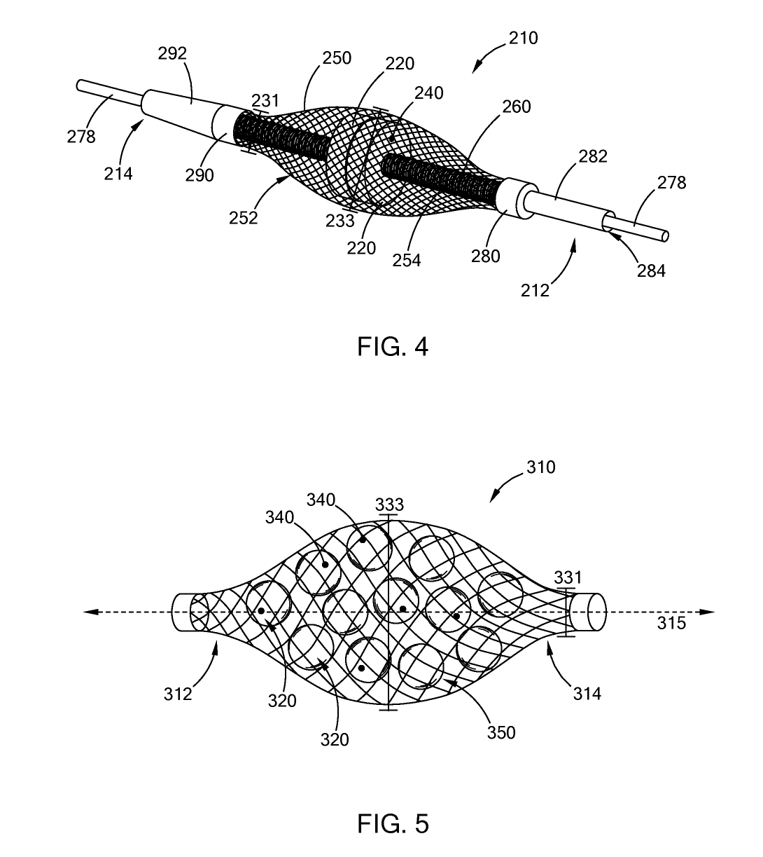

FIG. 4 shows another device in accordance with an embodiment of the present invention. In particular, device 210 has proximal end 212 and distal end 214 with longitudinal axis 215 running through the device. Other components are numbered similarly to components of FIG. 3 (for example, radiopaque members 240.)

Inner coil spring 260 runs from proximal collet 280 to distal collet 290, but unlike in the device of FIG. 3, the spring 260 herein consists of a single piece. As a consequence, when the device 210 is loaded into the delivery assembly, the spring is in an extended state; that is, there is a force of tension upon the spring, elongating it. As the device 210 is deployed to a body vessel of a patient, the tension force is release and the proximal end 212 moves toward the distal end 214 as the inner coil spring 260 contracts and decreases the longitudinal length of the device 210.

The device 210 optionally has a collet at each end. In the device illustrated in FIG. 4, the collets are of differing construction. It is to be understood that the designs described here could be employed at both ends if desired.

The proximal collet 280 has an extended portion 282 that is thinner than the body portion of the collet 280 which contacts the covering 250. This thinner extended portion allows for easier threading of a wire guide 278 through the lumen 284 of the proximal collet 280.

The distal collet 290 likewise has tapering end 292 distal to its main body portion which is in contact with covering 250. The tapering portion makes introduction to body vessels easier and allows for gentler navigation of tortuous portions of the anatomy.

As seen in FIG. 4, a wire guide 278 runs through the entire length of the device, substantially coinciding with longitudinal axis 215. This is possible because the device itself has an inner lumen, the inner coil spring 260 has an interior lumen, and there are holes at the proximal end of proximal collet 280 and the distal end of distal collet 290. These components are also hollow, allowing for the creation of wire guide lumen 284.

Elongated, flexible wire guides are used to gain access to specific inner areas of the body. The wire guide may enter the body through a small opening and travel to parts of the body through body channels. For example, wire guides may be passed through the body via peripheral blood vessels, gastrointestinal tract, or the urinary tract. Wire guides are commercially available and are currently used in cardiology, gastroenterology, urology, and radiology.

Once in place at a desired location in the body, wire guides are commonly used as guides for the introduction of additional medical instruments, e.g., catheters. A wire guide may have a proximal section coated with fluoropolymer coating, such as for example, a polytetrafluoroethylene (PTFE) coating. The lubricity of the fluoropolymer coating is sufficiently tactile to allow the interventionalist to feel wire movement.

Turning now to FIG. 5, another embodiment of a device in accordance with the principles of the present invention is illustrated. In this embodiment, the lumen 354 of device 310 is filled with expandable hydrophilic foam elements 320. In this case the covering 350 serves as the connecting member, and the foam elements 320 have a substantially spherical or rounded shape. There is no central inner coil spring that keeps the foam elements in alignment, allowing for freer expansion of the foam elements. Radiopaque members 340 are included in some or all of the hydrophilic foam elements 320. Depending on the shape of the vessel to which the device 310 is to be deployed, the first device height 331 may be significantly smaller than the second device height 333. However, these quantities may be substantially equal, or the second device height 333 may be less than first device height 331 under some conditions. The hydrophilic foam elements of this embodiment may be substantially spherical, or could be strips, or a combination of both configurations may be used.

In another embodiment, the expandable foam elements may take on different shapes. One example would be a substantially disc-shaped element that has been slitted, scored, dented, or otherwise manipulated in order to allow for collapse of the disc, such as how an umbrella would fold. This folding would allow for expansion and contraction in ways other than the usual radial expansion and may assist in easier packing into the delivery assembly, more flexibility in deployment, or both.

In one aspect, a device in accordance with principles of this invention may have a diameter of a disc-shaped hydrophilic foam element of about 8 millimeters to about 10 millimeters. In another aspect, such a device may have a total length from proximal end to distal end of about 10 millimeters to about 25 millimeters. The deployment of a device may be achieved with a catheter having a diameter of about 9 French, but may be achieved with catheter of about 6 French to about 10 French.

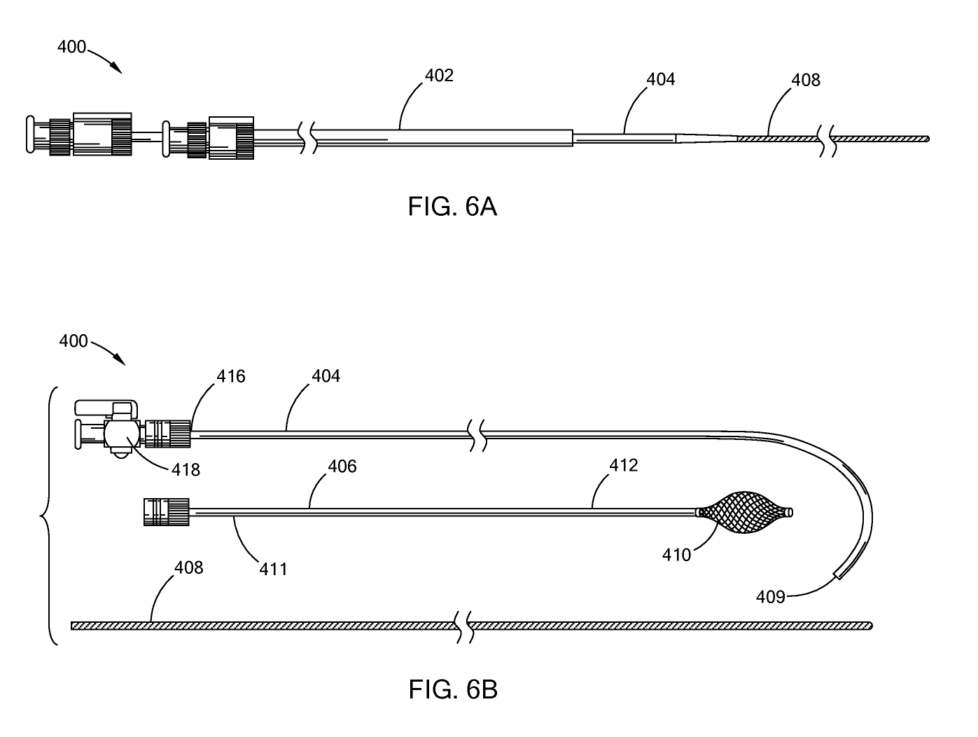

FIGS. 6A and 6B depict a delivery assembly 400 for introducing and retrieving any of the occlusion devices (designated in FIG. 6A-B with reference numeral 410) of FIGS. 1-5 for occluding a body vessel in accordance with some embodiments of the present disclosure. However, one skilled in the art will recognize that other delivery assemblies may be used for introducing and retrieving the occlusion device 410.

As shown, the delivery assembly 400 includes a polytetrafluoroethylene (PTFE) introducer sheath 402 for percutaneously introducing an outer sheath 404 into a body vessel. Of course, any other suitable material for the introducer sheath 402 may be used without falling beyond the scope or spirit of the present invention. The introducer sheath 402 may have any suitable size. The introducer sheath 402 serves to allow the outer sheath 404 and an inner member or catheter 406 to be percutaneously inserted to a desired location in the body tissue, cavity or vessel. The inner member may also include, for example, a stylet, or a modified pusher member which may include a mateable interface at its distal end for interacting securely and detachably with the proximal end of the occlusion device to be delivered. This mateable attachment may be, for instance, male and female components, or a nut and bolt configuration, or another configuration.

The introducer sheath 402 receives the outer sheath 404 and provides stability to the outer sheath 404 at a desired location of the body tissue, cavity or vessel. For example, the introducer sheath 402 is held stationary within the body tissue, cavity or vessel, and adds stability to the outer sheath 404, as the outer sheath 404 is advanced through the introducer sheath 402 into an opening. The outer sheath 404 has a body extending from a proximal end 416 to a distal end 409, the body being tubular and including a sheath lumen extending therethrough.

As shown, the assembly 400 may also include a wire guide 408 configured to be percutaneously inserted within the vasculature to guide the outer sheath 404 to the opening. The wire guide 408 provides the outer sheath 404 with a path to follow as it is advanced within the body tissue, cavity or vessel. The size of the wire guide 408 is based on the inside diameter of the outer sheath 404 and the diameter of the target opening.

When the distal end of the outer sheath 404 is at the desired location within the opening, the wire guide 408 is removed and the occlusion device 414, which may contact a distal portion 412 of the inner catheter 406, is inserted into the outer sheath 404. The inner catheter 406 is advanced (e.g. pushed) through the outer sheath 404 for deployment of the occlusion device 410 through the distal end 409 to occlude the opening. The catheter 2006 extends from a proximal portion 411 to a distal portion 412 and is configured for longitudinal movement relative to the outer sheath 404. In this example, the distal portion 412 is shown adjacent to the occlusion device 410 before introduction into the outer sheath 404. Thus, before deployment, the occlusion device 410 is coaxially disposed within the lumen of the outer sheath 404 and removably coupled (e.g. by a threaded capture the occlusion device 410) to the distal portion 412 of the catheter 406, or in the alternative, the occlusion device 410 is merely pushed by, but not coupled to, the distal portion 412 of the catheter 406.

The outer sheath 404 further has a proximal end 416 and a hub 418 to receive the inner catheter 406 and occlusion device 410 to be advanced therethrough. The size of the outer sheath 404 is based on the size of the body tissue, cavity vessel in which it percutaneously inserts, the size of the opening, and/or the size of the occlusion device 410.

In this embodiment, the occlusion device 410 and inner catheter 406 are coaxially advanced through the outer sheath 404, following removal of the wire guide 408, in order to position the occlusion device 410 to occlude the body vessel. The occlusion device 410 is guided through the outer sheath 404 by the inner catheter 406, preferably from the hub 418, and exits from the distal end 409 of the outer sheath 404 at a location within the opening. Thus, the occlusion device 410 is deployable through the distal end 409 of the outer sheath 404 by means of longitudinal relative movement of the catheter 406. In order to more easily deploy the occlusion device 410 into the body vessel, the occlusion device 410 may have a slippery coating, such as silicone or slipcoating. If the occlusion device 410 is self-expanding, the occlusion device 410 may self-expand from the radially collapsed state to the radially expanded state in response to, for example, temperature changes (e.g. if the occlusion device 410 is made of a nickel-titanium shape memory alloy), or for example, because the occlusion device 410 will return to its biased radially expanded state after being compressed in the outer sheath 404 in its radially collapsed state.

Likewise, this embodiment may also retrieve the occlusion device 410 by positioning the distal end 409 of the outer sheath 404 adjacent the deployed device in the vasculature. The inner catheter 406 is advanced through the outer sheath 404 until the distal portion 412 protrudes from the distal end 409 of the outer sheath 404. The distal portion 412 (e.g. which may include a snare) is coupled to a proximal end of the occlusion device 410 (e.g. to a retrieval member such as a hook or loop), after which the inner catheter 406 is retracted proximally, drawing the occlusion device 410 into the outer sheath 404.

A kit including the delivery assembly as described above may also include a device for crimping the occlusion device 410 down to a device diameter such that the device 410 can fit into the assembly as needed. Such crimpers, such as stent crimpers and multiple-wedge crimpers, are known in the art, but any apparatus capable of applying a radially-inward force to crimp the device may be utilized.

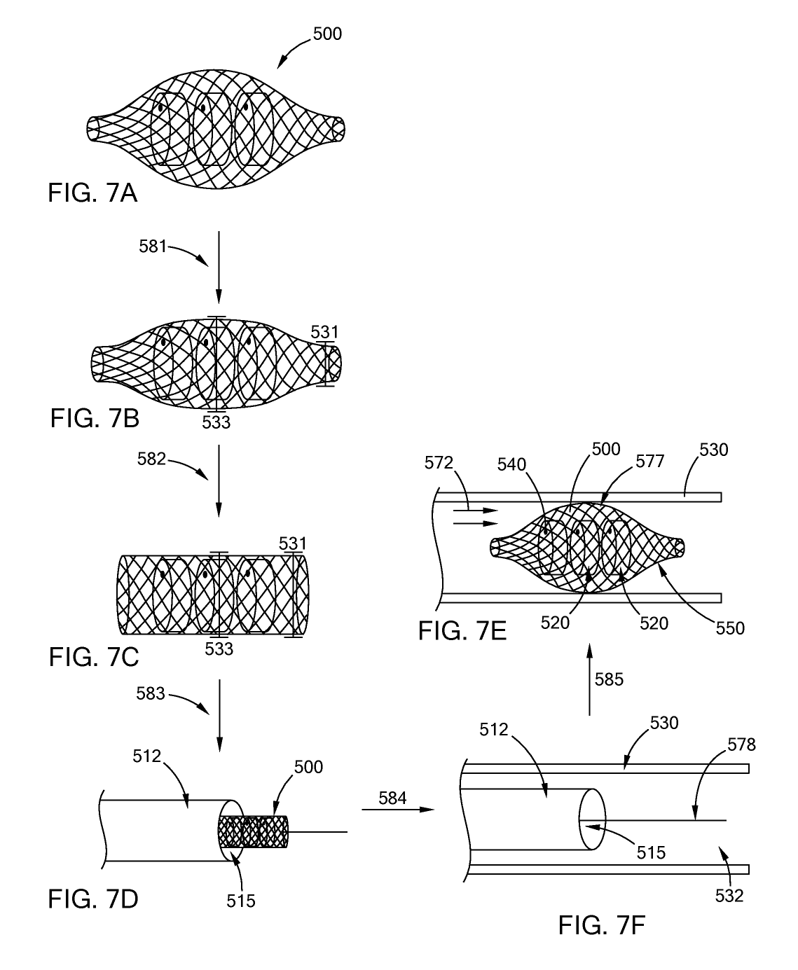

Turning now to FIG. 7, a method of loading the device into a delivery assembly and delivering the device to a body vessel is illustrated. In FIG. 7A, a device 500 is seen in its first, nonexpanded configuration. At step 581, the device is exposed to water or another aqueous solution in a wetting step. This causes the hydrophilic foam members to take up liquid and expand both radially and longitudinally. As illustrated, the members have expanded from their sizes in FIG. 7A to fill the covering more completely. The device of FIG. 7B has a first device height 531 at its end, and this height may be substantially equal at the proximal and distal ends. It also has a second device height 533 representing a maximum height for the device at the center, coincident with the height of the expanded hydrophilic foam element. The second device height 533 may be substantially equal to or greater than the first device height 531.

In a second step 582, the wetted device is crimped for loading into a catheter. FIG. 7C shows the result of crimping the occlusion device 500 using a crimping device. Force has been applied to the external portion of the device in the radial direction, causing the device to take on a substantially cylindrical shape. First device height 531 is now substantially equal to second device height 533. The crimping process causes some liquid to be squeezed out of the hydrophilic foam elements 520, so the device will be relatively dry after this step as compared to before the initiation of the crimping step.

In a third step 583, and as shown in FIG. 7D, the crimped device is loaded into a delivery assembly. The device 500 is loaded into the distal end of the outer sheath 512 of the delivery assembly and is taken into its lumen 515. Optionally, the crimped occlusion device may be loaded over wire guide 578 if it was constructed with, for instance, collets having lumens at its proximal and distal ends.

In a fourth step 584, and as shown in FIG. 7F, the delivery assembly is inserted into the body of a patient percutaneously. As shown, the distal end of the outer sheath 512 of a catheter has been inserted into the lumen 532 of body vessel 530 and wire guide 578 has been pushed through the catheter and into the vasculature of the patient to be treated.

In a fifth step 585, and as shown in FIG. 7E, the device is deployed to the vessel and occlusion is achieved. The device 500 has been expelled from the delivery assembly and now resides within the lumen 532 of the body vessel 530. In this illustration, the body vessel is a blood vessel. Blood flow 572 provides a liquid which is taken up by the hydrophilic foam elements 520, causing them to swell and occlude the vessel 530, the covering 550 being placed in contact with the vessel wall at contact point 577 due to the radial expansion of the hydrophilic foam elements 520 and, as a result, the device as a whole. Note that the illustration of FIG. 7E is not to scale for illustrative purposes and that in a true and operative device the foam discs 520 would expand to have substantially the diameter of the vessel they are shown as occluding.

In some instances it may be necessary to reposition or remove the closure device, particularly when it includes sufficiently flexible materials or a sufficiently flexible structural configuration. This may occur where the device is not appropriately positioned or sized for a particular bodily passageway and/or fails to completely seal the passageway. In cases where it is necessary or advisable to reposition the occlusion device following initiation of deployment or prior to full deployment, the practitioner may manipulate the delivery assembly such that the device is retracted partially or fully into the lumen 515 of the outer sheath 512 of the delivery assembly. The practitioner may then reposition the distal end of the delivery assembly and redeploy the device.

In certain embodiments, the occlusion device may optionally comprise thrombogenic material in order to encourage further occlusion. Suitable synthetic fibers include polyethylene terephthalate (DACRON), polyesters, polyamides (nylons), polyglycolic acid, polylactic acid, and the like. Other synthetic polymers having a lesser degree of thrombogenicity include fluorocarbons (Teflon) and polyaramids (Kevlar). Natural fibers such as silk and cotton are also suitable materials. The fibers may be attached to the device in any acceptable way. The thrombogenic material may be attached to the device by any suitable means, including tying, weaving, wrapping, or attaching by a silicone or other acceptable adhesive.

While the apparatus of the invention has been described above with reference to certain specific embodiments thereof, it is to be clearly understood that these embodiments have been given for purposes of illustration only and are not intended to be limiting. The scope of the invention is bounded only by the scope of the claims which are set out hereafter.

1. A device for occlusion of a body vessel, the device comprising:
a coil spring having a proximal end and a distal end, defining a longitudinal axis;
a proximal collet disposed to the proximal end and a distal collet to the distal end;
at least one expandable element disposed radially about a portion of the coil spring; and
a covering disposed on the proximal collet about the proximal end, the covering extending from the proximal collet about the coil spring and the at least one expandable element, the covering being disposed on the distal collet about the distal end.

2. The device of claim 1 wherein the hydrophilic foam elements are substantially disc-shaped.

3. The device of claim 1 wherein the covering comprises a mesh.

4. The device of claim 1 further comprising a threaded capture element extending proximally from the proximal collet substantially along the longitudinal axis.

5. The device of claim 1 wherein the coil spring defines a lumen formed therethrough along the longitudinal axis, each of the proximal collet and the distal collet comprising an opening formed along the longitudinal axis and in fluid communication with the lumen of the coil spring.

6. The device of claim 5 further comprising a tapered tip extending distally from the distal collet substantially along the longitudinal axis.

7. The device of claim 3 wherein the covering comprises a shape memory metal.

8. The device of claim 7 wherein the mesh further comprises a polyester.

9. The device of claim 8 wherein the shape memory metal and the polyester are present as strands, the strands being woven together to form the mesh, the shape memory metal strands and polyester strands being positioned in alternating fashion.

10. The device of claim 1 further comprising a radiopaque member embedded in the hydrophilic expandable foam member.

11. The device of claim 1 comprising at least two hydrophilic expandable foam members.

12. The device of claim 1 wherein the coil spring comprises:
a proximal coil spring half having a first end and a second end, the first end being attached to the proximal collet, the second end having a threaded member attached thereto and extending distally and substantially along the longitudinal axis; and
a distal coil spring half having a third end and a fourth end and defining a lumen formed therethrough, the fourth end of the distal coil spring being attached to the distal collet, the third end having a diameter such that the threaded member can enter the lumen threadedly.

13. An assembly for occlusion of a body vessel, the assembly comprising:
an outer sheath having a tubular body comprising an open end, the tubular body including a lumen formed through the open end;
an inner member disposed within the sheath lumen and movable rotationally and longitudinally relative to the outer sheath; and
an occlusion device comprising:
a coil spring having a proximal end and a distal end, defining a longitudinal axis;
a proximal collet disposed to the proximal end and a distal collet to the distal end;
at least one expandable element disposed radially about a portion of the coil spring; and
a covering disposed on the proximal collet about the proximal end, the covering extending from the proximal collet about the coil spring and the at least one expandable element, the covering being disposed on the distal collet about the distal end;
the occlusion device being disposed within the lumen and removably coupled to the distal end of the inner member and deployable through the open end of the outer sheath.

14. The kit of claim 13 further comprising a crimping device for crimping the occlusion device to a size to fit within the sheath lumen.

15. The kit of claim 13 further comprising a wire guide, and wherein the coil spring defines a lumen formed therethrough along the longitudinal axis, each of the proximal collet and the distal collet comprising an opening formed along the longitudinal axis and in fluid communication with the lumen of the coil spring, such that the wire guide can be passed through the lumen.

16. The kit of claim 13 wherein the device comprises a threaded capture element extending proximally from the proximal collet substantially along the longitudinal axis and the distal part of the outer sheath comprises a threaded receiving portion to threadedly engage the threaded capture element of the device.

17. A method of occluding a body vessel comprising:
wetting an occlusion device, the occlusion device comprising:
a coil spring having a proximal end and a distal end, defining a longitudinal axis;
a proximal collet disposed to the proximal end and a distal collet to the distal end;
at least one expandable element disposed radially about a portion of the coil spring; and
a covering disposed on the proximal collet about the proximal end, the covering extending from the proximal collet about the coil spring and the at least one expandable element, the covering being disposed on the distal collet about the distal end;
crimping the expandable element of the occlusion device to dispose the occlusion device within a lumen;
loading the occlusion device into the lumen;
introducing the delivery assembly percutaneously into the body vessel; and
delivering the occlusion device to the body vessel.

18. The method of claim 17 wherein delivering the occlusion device comprises advancing an inner member of a delivery assembly distally and rotating the inner member to release the occlusion device.

19. The method of claim 17 further comprising the steps of retracting the occlusion device back into the lumen, repositioning the lumen, and redeploying the occlusion device in the body vessel to be occluded.

20. The method of claim 18 wherein advancing the inner member precedes rotating the inner member.
